(19) United States
(12) Patent Application Publication
(10) Pub. No.: US 2018/0153213 A1
(43) Pub. Date: Jun. 7, 2018

(54) Vaporizer Metal Foam Filter

(71) Applicant: Porvair PLC, King's Lynn (GB)

(72) Inventor: Mark L. Heamon, Hendersonville, NC (US)

(73) Assignee: Porvair PLC

(21) Appl. No.: 15/832,386

(22) Filed: Dec. 5, 2017

Related U.S. Application Data
(60) Provisional application No. 62/430,719, filed on Dec. 6, 2016.

(51) Int. Cl. A24F 47/00; A24F 1/32; A61M 15/06; A61M 11/04

(52) U.S. Cl. CPC A24F 47/004; A24F 1/32; A61M 2205/3368; A61M 11/048; A61M 15/06

(57) ABSTRACT

An improved vaporizer for volalizing inhalants is described. The vaporizer comprises a heat source for heating air flowing through the vaporizer. A material comprising an inhalant is in the vaporizer and a metal foam filter is between the heat source and material. The heat source heats at least one of the air or metal foam filter to at least a vaporization temperature of the inhalant to below a combustion temperature of the material.

### CROSS-REFERENCE TO RELATED APPLICATIONS

The present application claims priority to pending U.S. Provisional Patent Application No. 62/430,719 filed Dec. 6, 2016 which is incorporated herein by reference.

### FIELD OF THE INVENTION

The present invention is related to a vaporizer or pipe, and more specifically, a vaporizer for tobacco, herbs and other materials suitable for inhalation. More specifically, the present invention is related to a metal foam filter, with secondary porosity, for use in a vaporizer.

### BACKGROUND

There has been a long-standing tradition of smoking plants and related materials wherein materials are burned and the smoke of combustion is inhaled. This tradition has been known to be used for inhaling the smoke of a myriad of materials including the smoke of tobacco, various herbs, and the like. It has long been established that the combustion products of such materials are harmful and, in many instances, the tradition of inhalation of combustion products has been vacated or significantly curtailed.

More recently, many of the desirable components of smoking materials have been isolated and utilized as a concentrate wherein the concentrate is vaporized for inhalation. The concentrate is preferably either a liquid or low melting solid which may be included in a solvent or matrix. By heating the materials to a vaporization temperature, but below the combustion temperature, the desired properties can be obtained without the inclusion of detrimental combustion products. This technique, commonly referred to as “vaping”, has revitalized those industries associated with inhalation products for public consumption due to reduced stigmatization associated with the use of vaporized materials relative to combustion materials.

The increased use of vaporized products has led to a desire for a vaporizer, also referred to in the art as a pipe, wherein the materials may be melted and/or vaporized at optimum temperature above the vaporization temperature but below the combustion temperature. There are many teachings of filters between the heat source and vaporization material but all are still ineffective. U.S. Pat. Nos. 9,445,629 and 7,434,584, both of which are incorporated herein by reference, teach a metal filter between the heat source and vaporization material wherein the metal filter is formed by granules of heat resistant materials bonded or sintered together. While somewhat effective, the filter functions as a radiant body thereby spreading heat and reducing the number of hot spots which can lead to combustion. Furthermore, the filter, once heated, can maintain sufficient heat to continue vaporization after the source of heating has been removed.

There is a strong desire for an improved filter suitable for use in a vaporizer, or pipe, wherein the vaporization of materials is more precise and combustion is minimized.

### SUMMARY OF THE INVENTION

It is an object of the invention to provide an improved device, or pipe, for vaporization of materials for inhalation.

It is another object of the invention to provide an improved vaporizer, or pipe, wherein vaporization is improved with minimal, and preferably no, combustion of the material being vaporized.

A particular advantage of the invention is provided by the use of a reticulated metal foam filter comprising inner channels, or voids, and microporosity wherein the inner channels or voids and microporosity are formed from vacated substrates and additives.

These and other embodiments, as will be realized, are provided in a vaporizer comprising a heat source for heating air flowing through the vaporizer. A material comprising an inhalant is in the vaporizer and a metal foam filter is between the heat source and material. The heat source heats at least one of the air or metal foam filter to at least a vaporization temperature of the inhalant to below a combustion temperature of the material.

### BRIEF DESCRIPTION OF THE DRAWINGS

FIG. 1 is a schematic representation of an embodiment of the invention.

FIG. 2 is a schematic representation of an embodiment of the invention.

FIG. 3 is a schematic representation of an embodiment of the invention.

### DESCRIPTION

The present invention is related to an improved vaporizer, or pipe, for vaporization of materials for inhalation. More specifically, the present invention relates to an improved porous, preferably reticulated, metal foam filter for use in an improved vaporizer or pipe for vaporization of materials for inhalation.

Metal foam filters are a specific type of reticulated material formed by a sponge replication technique wherein a porous organic substrate is impregnated with a metal precursor, preferably in a binder, wherein the precursor covers the interstitial struts of the organic substrate. The impregnated substrate is then heated to the extent necessary to sinter the metal and volatilize the organic substrate thereby providing a metal foam filter which is a replica of the original organic substrate with hollow portions therein representing that volume vacated by the volatilized organic substrate. Polyurethane in the form of an open-cell foam is particularly suitable for demonstration of the invention. The metal foam filter has a primary porosity which is approximately the porosity of the original organic foam used to form the metal foam filter with a preferred porosity of 10-130 ppi and more preferably 30-100 ppi. The pores are preferably interconnected and mostly open. The formation of metal foam filters is detailed in U.S. Pat. Nos. 8,202,346; 7,328,831; 6,773,825; 6,706,239 and 6,592,787 each of which is incorporated herein by reference.

In a particularly preferred embodiment a secondary porosity is provided wherein a volatile additive, such as organic spheres or polymer beads, is added to the metal precursor. Upon heating the volatile additive vaporizes resulting in a vacancy which approximates the size of the volatile additive thereby providing a secondary porosity which is significantly higher than the primary porosity. The secondary porosity is preferably in the range of 700 to 4500 ppi. The formation of secondary porosity is detailed in U.S. Pat. Nos. 6,235,665; 6,592,787 and 7,718,114 each of which is incorporated herein by reference.

Secondary porosity is preferably achieved by incorporating pore formers in the slurry wherein the pore formers are removed, preferably by vaporization during the heating steps. Particularly preferred pore formers are hollow, preferably organic, spheres and particularly hollow spheres. Hollow organic spheres are most preferred. The pore formers are chosen based on size with the understanding that the size of the pore formers is a closer approximation to the size of the eventual void. The porosity is easily controlled by the number of pore formers added and it is preferable that the pore formers are evenly dispersed and most preferably that each pore former is in contact with adjacent pore formers thereby creating a network of voids. Pore formers with an average diameter of 20 to 150 microns and more preferably 20-80 microns are suitable for demonstration of the invention.

In the preparation of the metal foam filter a slurry is prepared comprising powdered metal, binder and additives. The additives may include liquids, such as water or a solvent, shrinkage aids and pore formers. Exemplary binders include, without limit thereto, organic adhesives, starches, polyvinyl alcohol, acrylic binders, xantham gum, methylcellulose and phenolic binders. It is preferable that the binder be removed during heating, preferably, by vaporization. Shrinkage aids are preferably removed during the heating and lower the green density of the powdered metal thereby providing for additional shrinkage. Shrinkage aids are typically polymers. The slurry typically comprises about 0.1 to about 15 wt % binder, 0 to about 40 wt % liquid, 0 to about 5 wt % shrinkage aid and up to about 10 wt % pore formers with the balance being metal powder.

A foam is impregnated with the slurry preferably including multiple instances of compressing the foam in the slurry followed by allowing the foam to expand thereby insuring the interstitial struts of the foam are covered by slurry. The foam may be constrained into a predetermined shape through, at least, a temperature sufficient to dry the slurry and preferably through a temperature sufficient to sinter the metal.

The invention will be described with reference to the figures forming an integral non-limiting component of the disclosure.

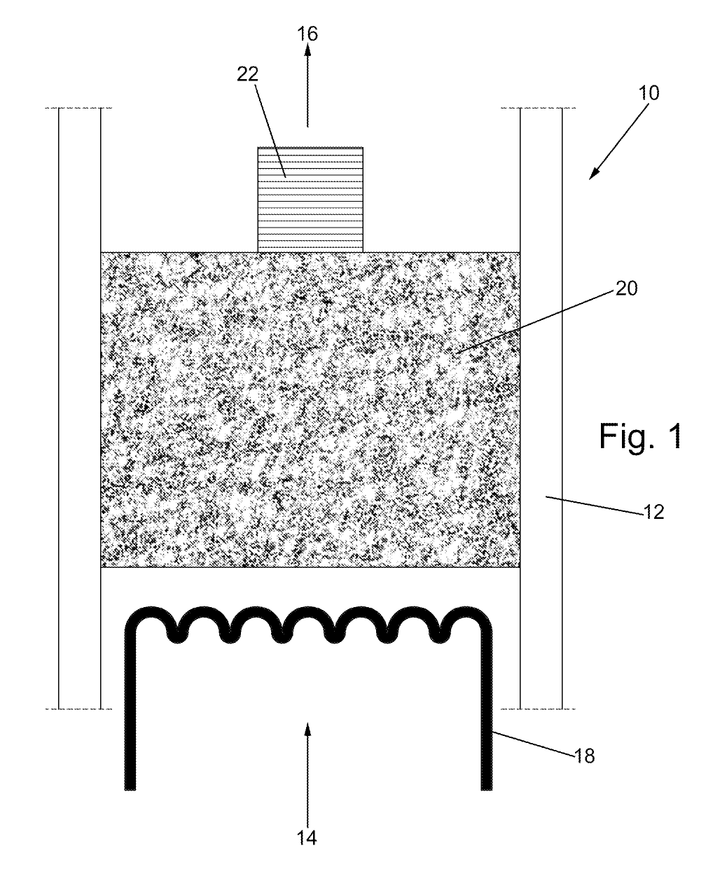

An embodiment of the invention will be described relative to FIG. 1 wherein the functional section of a vaporizer, 10, also referred to in the art as a pipe, is illustrated in partial cross-sectional schematic view. In FIG. 1, the vaporizer comprises a housing, 12, such as a tube the cross-sectional shape of which is not particularly limited. A material, 22, to be vaporized, such as a flower, is separated from a heating element, 18, by a, preferably reticulated, metal foam filter, 20. Air is drawn into the housing in the direction of arrow 14, past the heating element, wherein the air is heated. The heated air then flows through the metal foam filter thereby heating the metal foam filter. The heated air and radiant heat from the metal foam filter vaporize the volatile inhalant of the material wherein the volatile inhalant is entrained in the stream of air to form treated air, 16. The treated air is then inhaled by the user. There is no intended interaction between the material being vaporized, or the volatile inhalant, and the heat source.

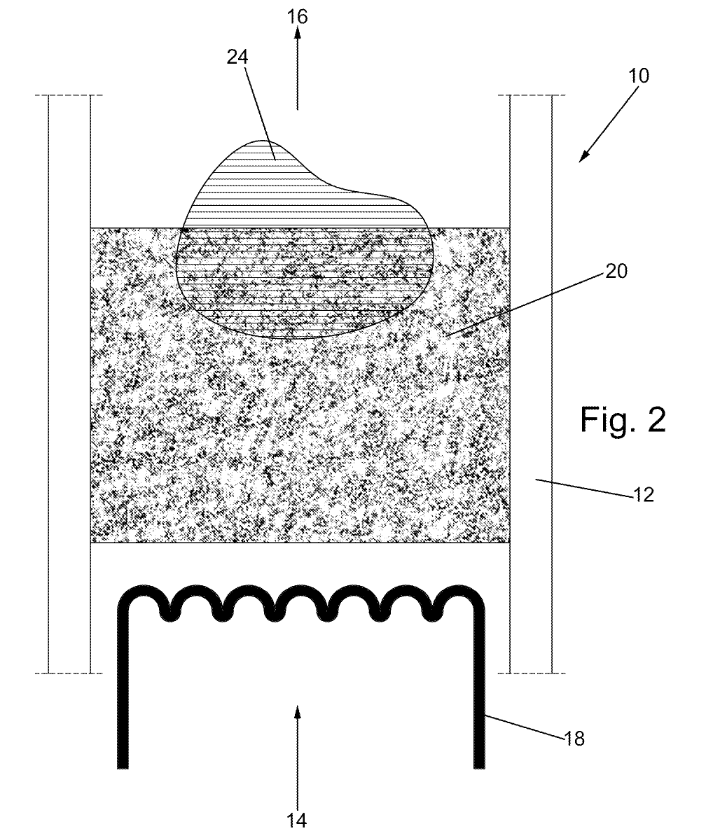

A particular advantage of the invention will be described with reference to FIG. 2 wherein the functional section of a vaporizer, 10, is illustrated in partial cross-sectional schematic view. In FIG. 2 the material is a concentrate, 24, comprising a material to be vaporized and an optional carrier such as a solvent or waxy material. It is preferable that the temperature of vaporization of the solvent or waxy material be higher than the temperature of vaporization of the desired inhalant of the material and that the combustion temperature of the solvent or waxy material be above the temperature reached by the vaporizer or metal foam filter. As the metal foam filter, and subsequently the concentrate, is heated the concentrate matriculates into the metal foam filter, presumably, due to capillary action due to the primary porosity and secondary porosity. The concentrate matriculating into the metal foam filter improves thermal conductivity to the material and improves the rate of vaporization thereby providing a more even evaporation of desired inhalants. Any remaining solvent or waxy material is captured by the metal foam filter for disposal with the metal foam filter of for removal by cleaning of the metal foam filter. The concentrates are placed near the metal foam filter or on the surface of the metal foam filter and as heat is applied the material melts and flows onto and into the surface area as air flows through the metal foam filter, either by natural inhalation or by pump assistance, thereby significantly improving vapor production efficiency with minimal energy consumption.

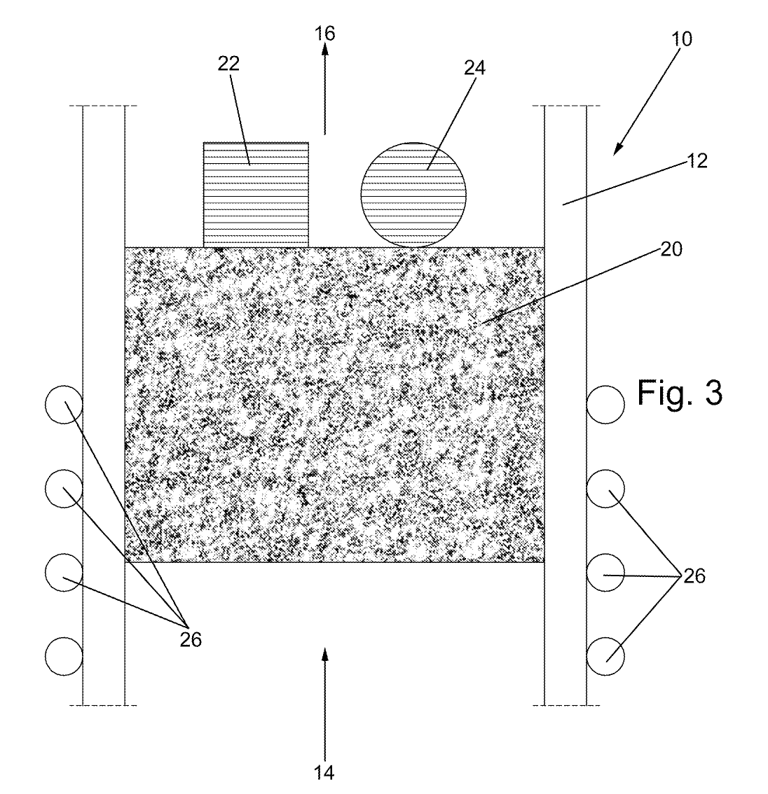

An embodiment of the invention is illustrated schematically in FIG. 3. In FIG. 3 the vaporizer is charged with a material, 22, and a concentrate, 24, for co-vaporization. A coil heating element, 26, on the exterior of the housing, 12, heats at least a portion of the housing containing the metal foam filter thereby heating the metal foam filter by thermal convection.

The metal used in the metal foam filter is preferably an oxidation resistant metal or metal alloy that readily allows sufficient heat transfer to heat the sample or concentrate such that the inhalant of interest vaporizes from the material. Particularly preferred metals, without limit thereto, includes stainless steel 314 (SS314), aluminum and copper. A particularly preferred metal foam filter is Metpore® Metal foam available from SELEE Corporation of Hendersonville, N.C.

The metal of the metal foam filter preferably has sufficient oxidation resistance at up to 600° F. in air.

The metal foam filter has a preferred relative density range of 3-15% as this range provides adequate material strength and heat transfer.

The metal foam filter can have solid struts, but hollow struts are hypothesized to significantly increase surface area and vaporization efficiency. The hollow struts draw in melted concentrates through capillary action into the hollow strut area thereby increasing the volume of concentrate material available for vaporization.

The metal foam filter can be compressed during heating whereby the starting ppi, referred to as the primary ppi, is compressed thereby increasing the surface area and vaporization efficiency.

The geometry and thickness of the metal foam filter is not particularly limited herein and is dictated by the design of the vaporizer which, other than allowing for the metal foam filter as an insert therein, is not otherwise limited herein. Sheets, disks, cylinders, hollow cylinders, or tubes are suitable for demonstration of the invention.

A particularly preferred metal foam filter for the purposes of demonstrating the invention comprises a rectangular Stainless Steel 314 with a size of approximately 6 inches by 6 inches with a thickness of 0.315 inches. A particularly preferred metal foam filter has a porosity of 40-90 ppi, and more preferably 60 or 80 ppi. A particularly preferred metal foam filter has a density of 3 to 7% of theoretical density and more preferably 4 or 6% of theoretical density.

The heating element is not particularly limited herein. The air flowing through the metal foam filter can be heated by conduction and by either direct heat or indirect heat. Resistive and infrared heating elements are most preferred due to their simplicity in operation and decreased potential for unintended combustion. The heating element is preferably a resistive heating element or an infrared heating element due to the ease of operation and control. A combustion based heating element, such as a flame generator, can be used but is less desirable. A temperature of between 300-600° F. is suitable for demonstration of the invention. A controller in communication with a detector, such as a thermostat or thermistor, may be employed to limit the active heating time when sufficient radiant heat is available from the metal foam filter.

Throughout the description porosity is defined using the units “ppi” which are standard in the art to indicate “pores per inch” or the number of pores, represented as a sphere, which aligned tangentially would span one inch. A metal foam filter with an 80 ppi porosity, for example, would indicate an average pore size of 1/80 inch equivalent diameter wherein equivalent diameter is the diameter of a circle having the same area as the cross-section of the pore.

Throughout the description density is represented as % relative density as is standard in the art wherein the density is reported as a percentage of the theoretical, or crystallographic, density of the material.

Though not limited thereto the present invention can be used with any volatile inhalation material of medical or recreational interest. Natural grown marijuana or synthetic produced marijuana, and there derivatives, or other psychoactive components are particularly suitable materials as are nicotine, flavors, medicines and other materials known to be suitable for inhalation as a vapor can be used independently or various inhalants may be blended. The material may be a plant or portion of a plant such as a flower or leaf and may be used as harvested or pulverized and mixed with inert elements or additional materials comprising preferred inhalants to be volatilized.

The invention has been described with reference to the preferred embodiments without limit thereto. Other embodiments and improvements will be realized which are not specifically stated but which are within the scope of the invention as set forth in the claims appended hereto.

Claimed is:

1. A vaporizer comprising:
a heat source for heating air flowing through said vaporizor;
a material comprising an inhalant; and
a metal foam filter between said heat source and said material wherein said heat source heats at least one of said air or said metal foam filter to at least a vaporization temperature of said inhalant to below a combustion temperature of said material.

2. The device of claim 1 wherein said heat source is selected from resistive and infrared.

3. The device of claim 1 wherein said material is selected from a flower and a concentrate.

4. The device of claim 3 wherein said material further comprises a solvent or waxy material.

5. The device of claim 1 wherein said metal foam filter comprises struts.

6. The device of claim 5 wherein said struts are hollow struts.

7. The device of claim 1 wherein said metal foam filter comprises primary porosity and secondary porosity.

8. The device of claim 7 wherein said primary porosity is 10-130 ppi.

9. The device of claim 8 wherein said primary porosity is 30-100 ppi.

10. The device of claim 9 wherein said primary porosity is 40-90 ppi.

11. The device of claim 7 wherein said secondary porosity is 700 to 4500 ppi.

12. The device of claim 1 wherein said metal foam filter comprises a metal selected from the group consisting of stainless steel, aluminum and copper.

13. The device of claim 12 wherein said metal is stainless steel 314.

14. The device of claim 1 wherein said metal foam filter has a density of 3-15% of theoretical density.

15. The device of claim 14 wherein said metal foam filter has a density of 3-7% of theoretical density.

16. The device of claim 14 wherein said reticulated metal filter has a density of 4-6% of theoretical density.

17. The device of claim 1 wherein said heat source heats at least one of said air or said metal foam filter to a temperature of at least 300° F. to 600° F.
